(19) United States
(12) Patent Application Publication
(10) Pub. No.: US 2022/0267290 A1
(43) Pub. Date: Aug. 25, 2022

(54) METHOD FOR EXTRACTION OF CANNABINOIDS AND SYNTHESIS OF THC TO CBN

(71) Applicant: Gaia Botanicals LLC DBA Bluebird Botanicals, Louisville, CO (US)

(72) Inventors: Tyler T D'Spain, Denver, CO (US); Melanie Hopek, Thornton, CO (US); Coleman Wenzl, Niwot, CO (US)

(21) Appl. No.: 17/180,033

(22) Filed: Feb. 19, 2021

(51) Int. Cl. C07D 311/80

(52) U.S. Cl. CPC C07D 311/80

(57) ABSTRACT

A method is disclosed to extract and purify cannabinoids from hemp biomass followed by isolation of various components including THC. The hemp-derived THC is reacted with a halogen, leading to the oxidation of THC to CBN. The removal of halogen is improved via selective solvent exchange. CBN is isolated from the reaction mixture by use of orthogonal chromatography techniques. The CBN is optionally recombined with the remaining purified cannabinoids to create a CBN enriched broad-spectrum extract.

### BACKGROUND OF THE INVENTION

### Field of the Invention

The present general inventive concept is directed to a method for extracting organic material from a biomass industrial hemp (Cannabis sativa L.) and selectively isolating the cannabinoids. The cannabinoid delta-9-tetrahydrocannabinol (THC) can be isolated and converted to cannabinol (CBN) and optionally recombined with the other extracted cannabinoids to provide a broad-spectrum composition that is free of THC.

### Description of the Related Art

The medical benefits of cannabis and cannabinoids have recently become more appreciated in the medical and scientific community. Several compounds present in hemp have been shown to have analgesic or anti-inflammatory benefits. The availability of hemp and cannabinoids free of THC has highlighted the benefits of this class of compounds as they are used by patients without the psychoactive effects of delta-9 tetrahydrocannabinol, or THC. Obtaining cannabinoids free of THC has been recently gaining more attention as the legal standing of industrial hemp in the USA has been altered. Hemp as defined as originating from a plant having less than 0.3% THC on a dry-weight basis has been removed from classification under the Controlled Substances Act. Manufactured products with less than 0.3% THC now enjoy a “legal” standing. Conversely, materials with a THC concentration greater than 0.3% on a weight basis are still illegal in some states and legal only under a prescription or doctor's recommendation in other states. Extraction of beneficial compounds from hemp biomass has been the subject of extensive scientific inquiry.

Numerous methods of extraction have been employed to extract materials from hemp biomass. U.S. Pat. No. 6,403,126 to Webster discloses an extraction method that utilizes a chromatographic column to fractionate various compounds individually. Isolation alone leaves numerous chemically similar substances to be discarded as waste and fractionation using column chromatography is solvent intensive, yield large amounts of hazardous waste. Numerous subsequent patent applications have been cited to the Webster patent. U.S. Pat. No. 9,950,976 to Keller discloses a method for extracting elements and then selectively recovering compounds from solvents. Some methods such as WO2016/004410 disclose the conversion of THC to CBN utilizing the addition of heat in the presence of oxygen over many days, paragraph [0025], and while the conversion is effective, the oxidative conditions are not selective for the conversion of THC to CBN. While some THC is converted to CBN, the process can take several weeks, limiting scalability, and may result in the oxidation of CBD to cannabielsoin (CBE) and cannabichromene (CBC) to cannabicyclol (CBL). Still other efforts such as U.S. Pat. No. 8,895,078 to Mueller provide a method of increasing THC content by converting CBD to THC.

As a matter of efficiency, it would be useful to selectively convert THC to a useful and legal analog, for example CBN, and produce a broad-spectrum cannabinoid composition of high purity that comports with the federal definition of “hemp.” Other approaches have been pursued, but result in residual THC in the final product, oxidize or degrade other components of the composition, waste large amounts of organic solvents, or are limited in their scalability.

U.S. Pat. No. 10,239,808 to Black discloses a method of obtaining CBD isolate from a stationary phase column. This approach obtains existing CBD concentrations present in the biomass without converting compounds through chemical synthesis.

What is needed is a low cost and scalable method of extracting organic compounds from a biomass to produce a high purity extract, and a method of selectively converting less desirable cannabinoids in a biomass to desirable cannabinoids to produce a product with minimal psychoactivity and high purity suitable for medical use or research.

### SUMMARY OF THE INVENTION

It is an aspect of the present invention to provide a method for extracting a high purity cannabinoid composition from a biomass. It is a further aspect of the present invention to provide a method for converting THC to CBN. It is a further aspect of the invention to provide a broad-spectrum cannabinoid composition from a biomass by extracting and purifying cannabinoids from a hemp biomass and isolating THC and converting THC to CBN and optionally recombining the CBN with the extracted, purified cannabinoids to provide a broad-spectrum cannabinoid composition of high purity.

The above aspects can be obtained by extracting cannabinoids from a biomass of hemp using an alcohol at a sub-freezing temperature, to yield a crude cannabinoid composition; heating of the crude cannabinoid composition to decarboxylate the cannabinoids in the extracted material and convert the natural carboxylic acid derivatives of cannabinoids to their active forms; distillation of the activated crude cannabinoid composition in two stages to remove undesirable components of the mixture, with the first stage removing components with a boiling point less than that of said cannabinoids, such as terpenoids and residual solvents to produce a first pass distillate; distillation of the first pass distillate by a second stage distillation to remove components with boiling points greater than said cannabinoids, such as pigments, lipids, and residual environmental contaminants such as lead and other heavy metals to produce a cannabinoid distillate; performing chromatography, e.g. centrifugal partition chromatography (CPC), to selectively isolate said cannabinoid distillate into a THC solvent fraction, and a broad spectrum extract in solvent fraction that is substantially free of THC; and drying said broad spectrum extract in solvent fraction to provide a broad spectrum cannabinoid mixture free of THC.

It is a further aspect of the invention to dry said THC in solvent fraction to produce a THC extract; dissolving said THC extract in a solvent; reacting said THC extract with a halogen to oxidize THC to CBN; quenching said halogen in an aliphatic solvent; and purifying CBY by orthogonal chromatographic purification to yield purified CBN. The method of the invention further provides combining the broad-spectrum cannabinoid mixture free of THC free of THC with said purified CBN to produce a CBN enriched broad spectrum cannabinoid composition. A final distillation step may be performed to further purify the component mixture and ensure the removal of solvents applied during the CBN synthesis step and reduce undesirable color from the CBN-enriched broad spectrum cannabinoid composition.

These together with other aspects and advantages which will be subsequently apparent, reside in the details of the method of the invention as more fully hereinafter described and claimed, reference being had to the accompanying drawings forming a part hereof, wherein like numerals refer to like parts throughout.

### BRIEF DESCRIPTION OF THE DRAWINGS

Further features and advantages of the present invention, as well as the structure and operation of various embodiments of the present invention, will become apparent and more readily appreciated from the following description of the preferred embodiments, taken in conjunction with the accompanying drawings of which:

FIG. 1 is a flow chart of a method in an embodiment of the invention.

FIG. 2 is a flow chart of products of the steps of a method in an embodiment of the invention.

### DESCRIPTION OF THE PREFERRED EMBODIMENTS

Reference will now be made in detail to the presently preferred embodiments of the invention, examples of which are illustrated in the accompanying drawings, wherein like reference numerals refer to like elements throughout.

The present inventive concept relates to a method of producing a broad-spectrum cannabinoid preparation from cannabis, cannabis sativa, or hemp. Disclosed herein is a method of extraction of cannabinoids from a biomass, separation of various chemical compounds, purification of compounds of interest, a novel conversion and purification method of THC to CBN, and further purification and analytical steps to determine and ensure purity followed by the option to recombine cannabinoids to produce a broad-spectrum cannabinoid composition of high purity.

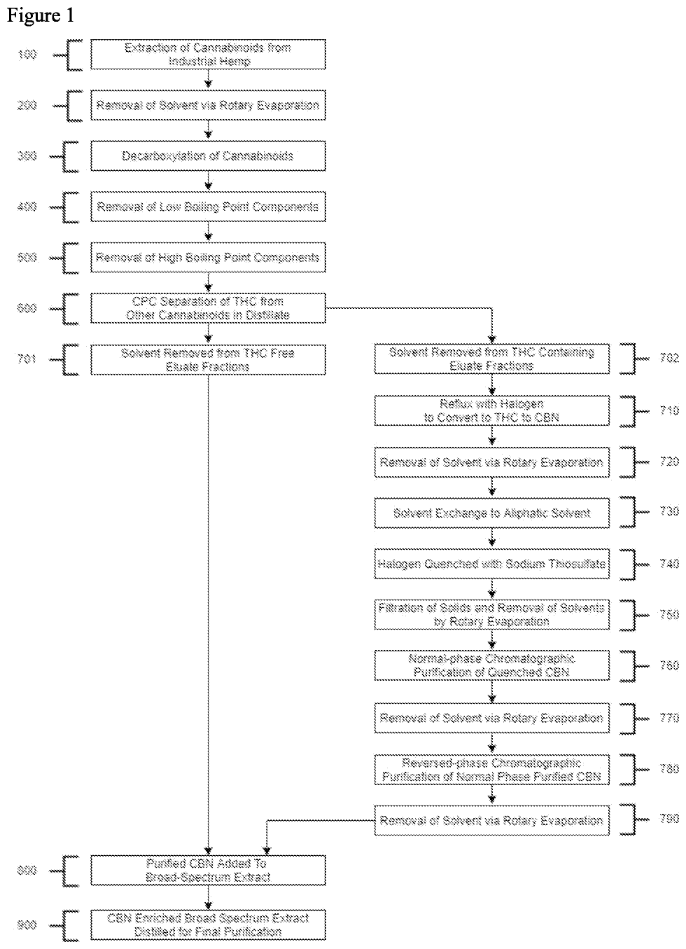

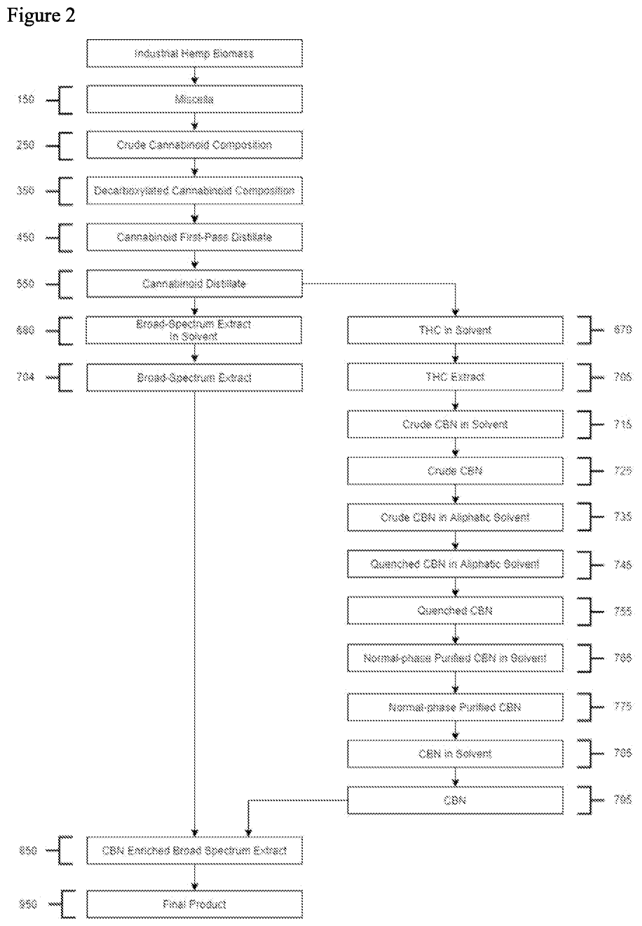

Detailed description of the method of the invention will be made with reference to FIGS. 1 and 2. FIG. 1 presents a flow chart of the method steps in an embodiment of the invention. FIG. 2 presents a flow chart of the products from each step of the method. Preparation of biomass is conducted by selection of a desired plant. Dried plant material can be provided and can be chopped or crushed to increase workability and surface area.

In extraction step 100, a biomass can be placed into an extraction column and extracted with a solvent. A cold solvent, for example an alcohol, can be utilized in an embodiment of the invention. In one such embodiment, ethanol denatured with 5% n-heptane at a temperature of approximately −40° C. can be flowed into a biomass extraction vessel and allowed to saturate and extract the biomass for 12 to 15 minutes before being transferred to a collection vessel. The resultant mixture, considered miscella 150, may be passed through a fine mesh filter to remove solid particulate. In extraction evaporation step 200, the solvent may be removed from the filtered miscella to yield a crude cannabinoid composition 250. In one such embodiment, the filtered miscella is transferred to a 20-liter rotary evaporator and heated to around 50° C. under vacuum to yield a crude cannabinoid composition 250. The crude cannabinoid composition 250 contains a mixture of dissolved organic compounds including, but not limited to, cannabinoids, terpenoids, flavonoids, and lipids.

In heating step 300, decarboxylation is advantageously employed at this stage to convert carboxylic acid derivative compounds to their neutral analogs, including THC-A to THC. This can be accomplished by heating the crude cannabinoid composition 250. Varying temperature and durations are effective, for example the extract can be heated to 120° C. under vacuum in a Pyrex dish in an electric vacuum oven for 30 minutes. The heating step 300 of the method can include infrared heat lamps or other known heating mechanisms. Cannabinoid acids are converted to neutral cannabinoids through heating and the byproduct is carbon dioxide. The mechanism is shown below.

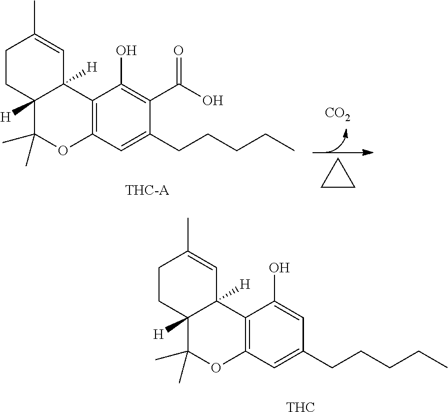

The result is a decarboxylated cannabinoid composition 350 that appears as an opaque dark green to brown viscous oil.

Distillation can then be employed to the decarboxylated cannabinoid composition 350 to achieve a concentrated fraction of cannabinoids. In one example, the decarboxylated cannabinoid composition 350 is loaded into a wiped film evaporator for molecular distillation under vacuum. An explanation of single stage wiped film distillation apparatus is detailed in Distillation of Natural Fatty Acids and Their Chemical Derivatives by Cermak, Steven & Evangelista, Roque & Kenar, James. (2012) 10.5772/38601. In a preferred embodiment, two distillations are performed in tandem using a Precision Extraction CDU 1000 commercial distillation system. In first pass distillation step 400, the volatile components such as terpenes, residual solvents, and other volatile compounds are removed from the decarboxylated cannabinoid composition 350. In one embodiment of first pass distillation step 400, the evaporator body is set to 135° C. and the condenser is set to 25° C., and the system is operated under vacuum in the range of 0.1 to 0.3 torr. The decarboxylated cannabinoid composition 350 is fed into the evaporator body at a rate of 30 mL per minute and the wiper is operated at 380 rpm. The collected product on the residue collection side is considered cannabinoid first-pass distillate 450.

Distillation is repeated in second pass distillation step 500 upon cannabinoid first-pass distillate 450, to separate the higher boiling point fraction, which aids in the removal of pigments, such as chlorophyll, lipids, and other undesirable byproducts of the extraction process. In an embodiment of step second pass distillation step 500, the evaporator body is set to 175° C. and the condenser is set to 85° C., and the system is operated under vacuum of 0.005 to 0.05 torr. The cannabinoid first-pass distillate 450 is fed into the evaporator body at a rate of 25 mL per minute and the wiper is operated at 380 rpm. The product collected on the distillate collection side is considered cannabinoid distillate 550. The material collected on the residue collection side is waste.

Once the cannabinoids have been substantially isolated, further purification and separation can be advantageously achieved through chromatographic separation in CPC chromatography step 600. CPC is utilized for this step due to the significantly decreased solvent usage when compared to column or flash chromatography. Although the resolution of the CPC chromatograph is less than an equivalent scale flash chromatography system, the resolution of CPC is found to be sufficient for the removal of THC and the significant cost savings in both solvents and silica gel columns makes CPC an advantageous technique. The cannabinoid distillate 550 can be diluted in an alcohol, such as methanol, to decrease viscosity and then separated via centrifugal partition chromatography (CPC) preferably with a polar stationary phase and a non-polar mobile phase. Suitable equipment includes, for example, the Gilson CPC 1000 Pro and other similar equipment can provide similar results. In one example the polar stationary phase comprises methanol and water and the non-polar mobile phase comprises heptane. Straight chain alkanes with a number of carbon atoms ranging from five to eight can be utilized for the mobile phase in the CPC chromatography step 600 of the method. Fractions are collected and analyzed by high performance liquid chromatography with diode array detection (HPLC-DAD) to confirm purity, then the THC-free cannabinoid containing fractions are combined to provide broad spectrum extract in solvent 680; the THC-containing fractions are separately combined to provide THC in solvent 670. The solvent is removed from the solution of THC in solvent 670 by THC drying step 702 comprising rotary evaporation to yield THC extract 705. The solvent is removed from the broad-spectrum extract in solvent 680 by broad-spectrum drying step 701 comprising rotary evaporation to yield broad-spectrum extract 704. The products are a broad-spectrum extract 704 comprising cannabinoids derived directly from the natural distribution present in the biomass, and a THC extract 705. The broad-spectrum extract is free of THC and suitable for medical use and complies with laws prohibiting the presence of THC.

THC is a complex molecule that is expensive to synthesize. As the analogous compound CBN is known to have sedative and sleep-promoting properties and may possess analgesic and anti-inflammatory properties another aspect of the invention provides conversion of the THC to CBN to provide either a pure CBN composition or alternately combine the CBN with the broad-spectrum extract of the previous step to provide a CBN-enriched broad-spectrum extract.

The conversion of THC in a pure form or in combination with other related compounds or impurities can be accomplished in a synthesis comprising the following steps. In synthesis step 710, THC extract 705 can be reacted with a halogen, e.g. iodine, in an aromatic solvent, e.g. toluene, at an elevated temperature. The exact mechanism is unknown, but most likely consists of a series of addition/elimination reactions of halogen (e.g. iodine) across available double bonds. Upon elimination, a hydrogen halide is formed through the loss of a hydrogen from the cannabinoid. The addition/elimination of halogen across the double bonds continues until the hydrocannabinols such as THC are fully oxidized to cannabinol. The reaction temperature and duration may be modified to vary the yield and side products of the reaction. In a preferred embodiment, the reaction mixture of synthesis step 710 is subject to reflux at approximately 110° C. for 10 to 60 minutes. Preferably, the reflux is conducted between 30 and 45 minutes.

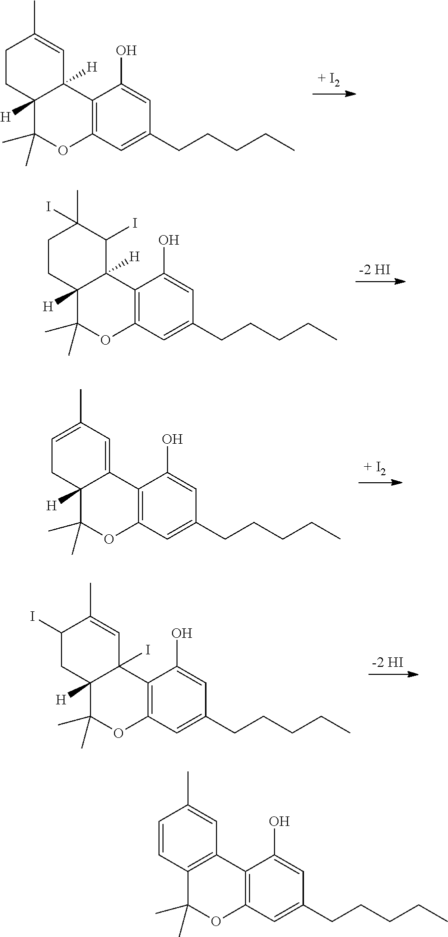

The halogen utilized in the conversion may consist of bromine, chlorine, or iodine, in pure form. In a preferred embodiment, iodine is utilized as the halogen. Bromine and chlorine are alternatives, but the volatility of these halogens increase the complexity of the required apparatus. The halogen may be provided with a purity of 99.5% (w/w) or greater to reduce the complexity of the reaction mixture and simplify the purification. In various embodiments, the halogen may be provided at about 1 to 3 moles of molecular halogen for every mole of THC. If too little halogen is used, the reaction yield will be reduced. If too much halogen is used, the rate of the reaction will increase, but at increased cost and complexity of reaction by-products that may be degraded by excess halogen amounts.

After the reaction has progressed for the desired duration, crude CBN in solvent 715 is achieved. The solvent can be removed in product drying step 720 which can comprise rotary evaporation under reduced pressure and elevated temperature yielding the crude reaction product, crude CBN 725. Solvent removal also provides the benefit of removing volatile colored reaction products, turning the recovered solvent pink.

To promote the removal of residual halogen, solvent exchange step 730 can comprise dissolving crude CBN 725 in a non-polar, aliphatic solvent, which reduces the solubility of the halogen to produce crude CBN in aliphatic solvent 735. The non-polar solvent may be either pentane, hexane, cyclohexane, heptane, or any mixture thereof. The exchange of solvent from toluene to an aliphatic hydrocarbon is critical to ensure the removal of halogen enumerated in the following steps.

To remove unreacted halogen from the crude product, quenching step 740 can comprise washing the solution of crude CBN in aliphatic solvent 735 with a saturated solution of sodium thiosulfate, such that a minimum of 2 moles of thiosulfate are used per mole of halogen. In one such example, sodium thiosulfate reacts with iodine to form sodium iodide, which is water-soluble and can be removed by liquid-liquid extraction. In the liquid-liquid extraction, the aqueous phase is partitioned by gravity separation and removed resulting in the removal of most of the sodium thiosulfate and sodium iodide. The organic phase may be further processed with the addition of brine, a saturated solution of sodium chloride in water. The mixture is shaken vigorously then the aqueous and organic phases are allowed to separate naturally. Once separated the brine solution is removed, leaving the organic phase free of excess halogen, sodium thiosulfate, and water. Optionally, the organic phase may be dried further with the addition of sodium sulfate or magnesium sulfate, then filtering the solution to remove the sodium or magnesium sulfate to yield quenched CBN in aliphatic solvent 745.

In quenched CBN drying step 750, the solvent is removed by rotary evaporation yielding quenched CBN 755, with a CBN concentration ranging from 15% (w/w) to 30% (w/w). A temperature no less than 10° C. below the solvent's boiling point while operating under vacuum of 50 torr or lower ensures the full removal of the non-polar solvent. At this point in the method, the reaction is complete and a significant portion of the THC in THC extract 705 has been converted to CBN in quenched CBN 755. However, reaction side products are present and are preferably removed to provide pure CBN.

The quenched CBN 755 may be purified using an orthogonal succession of normal phase chromatography step 760 followed by reversed phase flash chromatography step 780. It has been discovered that a sequence of chromatography utilizing a polar stationary phase followed by chromatography utilizing a non-polar stationary phase can provide a multistep purification process with excellent results. In particular, normal phase chromatography may be performed in normal phase chromatography step 760, for example, by chromatography using silica-gel as the stationary phase and a solvent selected from the group of pentane/hexane/heptane or petroleum ether in combination with ethyl acetate as the mobile phase. Chromatography using silica-gel media provides removal of highly polar and colored byproducts that are trapped by to silica gel. These are likely halogenated intermediates, and other reaction byproducts. The eluate, normal phase purified CBN in solvent 765, is collected as one fraction, which is dried in eluate drying step 770 comprising rotary evaporation under vacuum with a temperature between 75-85° C. to yield normal phase purified CBN 775.

Reversed phase flash chromatography step 780 can comprise reversed phase flash chromatography performed using C18 coated silica-gel as the stationary phase with ethanol or methanol mixed with water as the mobile phase. Mobile phase gradient programming can be used to control the eluent strength throughout the run. Reversed phase chromatography effectively separates any unreacted THC, or other impurities from the normal phase purified CBN 775, yielding CBN in solvent 785. The eluate is collected in 25 mL fractions, which are tested by HPLC-DAD to confirm purity. A small sample of the fraction is diluted 200-fold with methanol, filtered using a PTFE syringe filter, and analyzed. The peak purity is determined at 228 nm. All fractions with peak purity of CBN greater than 95% are combined to provide CBN in solvent 785. To make the CBN easier to recover from the eluate, CBN drying step 790 can comprise a liquid-liquid extraction to transfer the CBN from the water and methanol solution to a non-polar organic solvent. To improve phase separation between the non-polar solvent and the water and methanol solution, the methanol may be removed by rotary evaporation under vacuum until the solution turns milky white. The non-polar solvent, pentane/hexane/heptane or petroleum ether is added to extract CBN. After the two solvents are shaken vigorously, the aqueous and organic phases are allowed to separate. The aqueous phase is drained to waste, and the organic phase is collected. To ensure the organic phase is free of residual water, the solution may optionally be dried with magnesium or sodium sulfate, filtered to remove the magnesium or sodium sulfate. The solvent may be removed in the final stage of CBN drying step 790 to yield CBN 795. Rotary evaporation is performed below 0.05 Torr and 75-85° C. to ensure full removal of solvents. In various embodiments, CBN drying step 790 provides CBN 795 at a purity of 95% to greater than 99%.

The purified CBN 795 is a unique and valuable product on its own, which may be used as-is or may be recombined in mixing step 800 with the THC free broad-spectrum extract 704 which yields a unique product, THC free CBN enriched broad spectrum extract 850. The CBN enriched broad spectrum extract 850 is unique it that it contains a natural profile of cannabinoids while selectively replacing THC, a potent psychoactive compound with a substantially less potent analog, while at the same time making the product compliant with federal regulations regarding THC content in hemp products. Optionally, a last purification step may be performed to ensure that the final product 950 is free of volatile solvent residue left over from the chromatography steps 600 and 780. The CBN enriched broad spectrum extract 850 may be distilled in final distillation step 900 using the distillation parameters from second pass distillation step 500 to collect the solvent free CBN enriched broad-spectrum extract as the final product 950.

The following examples present various aspects of the invention, illustrating advantages obtained by the present invention compared to alternative methods. As used herein, the term “about” refers to a +/−variation of 10% from any given value. It may be understood that variation is always included in a given value unless provided herein, whether or not it is specifically referred to. All chemicals, reagents, and solvents used in the present examples were purchased from Sigma-Aldrich and used as-is unless specified otherwise.

### Example 1: Preparation of CBN

Synthesis step 710 comprises a starting compound of 16 g of THC extract, approximately 80% THC w/w. In this example the starting material THC extract 705 was isolated from cannabinoid distillate produced by CBD CliniLabs of Denver, Co. and synthesis step 710 comprised dissolving the THC in 400 ml of toluene followed by addition of 25.9 g of iodine. The solution was allowed to reflux with stirring at about 110° C. for 45 minutes. The solution was then allowed to cool to room temperature, about 23° C., to yield crude CBN in solvent 715. Product drying step 720 comprised filtration and drying by rotary evaporation at about 45° C. and 5 to 50 ton leaving a dark viscous oil, crude CBN 725. Solvent exchange step 730 comprised crude CBN 725 dissolved in n-heptane (400 mL) from TDA Chemical to make crude CBN in aliphatic solvent 735. The crude CBN in aliphatic solvent 735 was then washed with a saturated solution of sodium thiosulfate in quenching step 740 (2 L proportioned over 5 consecutive washes). The mixture was washed with a brine wash (400 mL) of saturated sodium chloride in water. The brine was drained and discarded, then the solution was dried with sodium sulfate (10 g) and filtered to remove solids. Quenched CBN drying step 750 further comprised rotary evaporation, yielding quenched CBN 755 (15 g, 15-30% w/w).

### Example 2: Purification of CBN

Normal phase chromatography step 760 was conducted with the quenched CBN 755 (15 g) dissolved in heptane (15 mL) and purified by vacuum normal phase chromatography by loading the mixture onto silica gel (100 g) and eluted with 9:1 heptane/ethyl acetate (1000 mL). The entire eluate consisting of CBN, the tetrahydrocannabinols and other similar impurities is collected which yields normal phase purified CBN in solvent 765. The silica removes highly polar contaminants from the quenched CBN. In eluate drying step 770, the normal phase purified CBN in solvent was dried by rotary evaporation yielding normal phase purified CBN 775 (12.5 g, 25-30% w/w).

Reversed phase chromatography step 780 was conducted with the normal phase purified CBN 775 (12.5 g) dissolved in dimethylformamide (DMF) (12.5 mL) and purified by reversed phase flash chromatography on an 1850 g C18 coated silica gel flash cartridge, Biotage SNAP KP-C18-HS. Utilizing gradient programming, the mixture was separated by gradient elution of methanol-water from 75% to 95% methanol controlled by gradient programming over 65 minutes with a flow rate of 500 mL/min. The eluate was collected in 25 mL fractions. The CBN containing fractions were analyzed by HPLC-DAD to confirm purity. The CBN containing fractions with a purity greater than 95% were combined to provide CBN in solvent 785. In CBN drying step 790, the methanol was removed by rotary evaporation until the mixture turned milky white, then heptane (500 mL) and brine (250 mL) were added and the solution was mixed vigorously, then allowed to separate in a 2 L glass separatory funnel. The aqueous layer was drained, then sodium sulfate (10 g) was added followed by mixing, then the solution was filtered to remove the sodium sulfate. The organic phase was dried by rotary evaporation, yielding CBN 795 (2.5 g, 95-100% w/w). The purified CBN 795 can be used as-is or may be recombined with the THC free broad-spectrum extract 704 in mixing step 800 to create THC free CBN enriched broad spectrum extract 850. Optionally the combined product 850 may be distilled in final distillation step 900 to ensure the final product is free of volatile solvent impurities. For final distillation step 900, the distillation parameters used in second pass distillation step 500 are repeated to collect the solvent free CBN enriched broad-spectrum extract as the final product 950.

Analysis of purity in CBN in solvent 785 and CBN 795 were determined by high performance liquid chromatography with a diode array detector and quadrupole mass spectrometer (HPLC-DAD-MS). Compound identities were confirmed by their retention times and mass-to-charge ratios. Mass-to charge ratios were determined on the same instrument running positive-mode electrospray ionization mass spectrometry (ESI-MS). The separations were performed on an end-capped C18 column by gradient elution with water (10 mmol ammonium formate, pH 3.6) and acetonitrile containing 0.1% formic acid. Purity was determined at 228 nm by peak area %. The most prevalent impurities from the synthesis were determined to be tetrahydrocannabinol isomers by their mass-to-charge ratios. The final product 950 obtained from the method of the invention is free of THC and suitable for medical use and complies with laws prohibiting the presence of THC.

Any description of a component or embodiment herein also includes hardware, software, and configurations which already exist in the prior art and may be necessary to the operation of such component(s) or embodiment(s). Further, the operations described herein can be performed in any sensible order. Any operations not required for proper operation can be optional.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

Further, the operations described herein can be performed in any sensible order. Any operations not required for proper operation can be optional.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

What is claimed is:

1. A method of isolating, modifying, and recombining components of industrial hemp to produce a cannabinoid extract free of THC and rich with other cannabinoids comprising the steps of:
extracting hemp with an alcohol to produce a crude cannabinoid composition;
heating the crude cannabinoid composition to decarboxylate cannabinoids;
distillation of the cannabinoids to remove low boiling point impurities and provide a first pass distillate;
distillation of said first pass distillate to remove high boiling point impurities and provide a cannabinoid distillate;
performing chromatography to isolate said cannabinoid distillate into a THC in solvent fraction and a broad-spectrum extract in solvent fraction; and
drying said broad spectrum extract in solvent fraction to provide a broad-spectrum cannabinoid mixture free of THC.

2. The method of claim 1 further comprising the steps of:
drying said THC in solvent fraction to produce a THC extract;
dissolving said THC extract in a solvent;
reacting said THC extract with a halogen to oxidize to CBN;
quenching said halogen in an aliphatic solvent; and
purifying CBN by orthogonal chromatography to yield purified CBN.

3. The method of claim 2 further comprising the steps of:
combining a broad-spectrum cannabinoid mixture free of THC.
combining said broad spectrum cannabinoid mixture free of THC and said
purified CBN to produce a CBN enriched broad spectrum extract.

4. The method of claim 3, wherein said solvent is toluene.

5. The method of claim 4, wherein said halogen is iodine.

6. The method of claim 5, wherein the reaction mixture is solvent exchanged from toluene to an alkane after the reaction is complete to decrease soluble iodine.

7. The method of claim 6, wherein said alkane is heptane.

8. The method of claim 1, wherein said chromatography comprises centrifugal partition chromatography (CPC) performed on a stationary phase of water and methanol with heptane mobile phase.

9. The method of claim 2, wherein said orthogonal chromatography comprises a normal phase chromatography performed on a silica gel stationary phase with heptane-ethyl acetate mobile phase.

10. The method of claim 9, wherein said orthogonal chromatography step further comprises a reversed phase chromatography performed on C18 silica gel with methanol-water mobile phase.

11. The method of claim 10 wherein said reversed phase chromatography is performed after said normal phase chromatography.
